(19) United States
(12) Patent Application Publication
(10) Pub. No.: US 2002/0034501 A1
(43) Pub. Date: Mar. 21, 2002

(54) Methods and compositions for promoting angiogenesis using monocytes

(76) Inventors: Robert Pawliuk, Medford, MA (US); Philippe Leboulch, Charlestown, MA (US)

Correspondence Address: LAHIVE & COCKFIELD, 28 STATE STREET, BOSTON, MA, 02109, US

(21) Appl. No.: 09/860,657

(22) Filed: May 18, 2001

Related U.S. Application Data
(60) Provisional application No. 60/205,063, filed on May 18, 2000.

(51) Int. Cl. A61K 48/00

(52) U.S. Cl. 424/93.21; 424/93.7

(57) ABSTRACT

Novel compositions and methods for treating myocardial and peripheral ischemia are disclosed which employ monocytes to provide localized, controlled doses of secreted therapeutic proteins to selected tissue areas. These proteins can be naturally produced my monocytes, or produced following genetic transduction of monocytes or their progenitor cells with appropriate expression vectors.

### RELATED INFORMATION

This application claims priority to provisional patent application U.S Serial No. 60/205,063, filed on May 18, 2000, incorporated herein in its entirety by this reference. The contents of all patents, patent applications, and references cited throughout this specification are hereby incorporated by reference in their entireties.

### BACKGROUND OF THE INVENTION

The depletion of oxygen supply to due to obstructed or inadequate blood supply is the common pathological state associated with tissue ischemias, including myocardial ischemia, ischaemic bowel disease, and peripheral ischemia. The alleviation of tissue ischemia is critically dependent upon angiogenesis, the process by which new capillaries are generated from existing vasculature. The spontaneous growth of new blood vessels provide collateral circulation surrounding an occluded area, improves blood flow, and alleviates the symptoms caused by the ischemia. Although surgery or angioplasty may help to revascularize ischemic regions in some cases, the extent, complexity and location of the arterial lesions which cause the occlusion often prohibits such treatment.

An alternative approach for the treatment of tissue ischemia has been the use of proteins which stimulate angiogenesis. Methods which have been investigated involve the injection of purified recombinant proteins and/or expression vectors which encode such factors. However, safety, efficacy and persistance of transgene expression conspire to limit this approach. The successful treatment of tissue ischemia requires sustained expression of a secreted protein for a specific period of time (i.e., a few months). Expression which is prematurely terminated provides an insufficient therapeutic effect, while permanent expression can be dangerous. The well-characterized non-integrative expression vectors, including adenoviruses, adeno-associated virus (AAV), naked DNA and liposomes, provide only transient expression and are therefore not applicable. Conversely, the integrative nature of other vectors, such as retroviruses and lentiviruses, are able to supply protein expression for the life span of the transduced cell, thus adding risk to their use. Although inducible expression systems have been developed (for example, tetracycline-responsive expression vectors), these systems have yet to be characterized for therapeutic uses in vivo.

Accordingly, improved methods for safe, controlled delivery of therapeutic proteins to localized tissue areas would greatly benefit patients suffering from tissue ischemia and other pathologies.

### SUMMARY OF THE INVENTION

The present invention provides novel methods and compositions for promoting angiogenesis within selected localized areas of tissue. In all embodiments, the invention employs monocytes, either purified from natural sources or recruited endogenously to localized areas of tissue (e.g., using chemoattractants), to mediate angiogenesis by delivering high, localized concentrations of secreted therapeutic proteins, either normally produced by monocytes or which are produced by monocytes following genetic transduction, in amounts effective to induce angiogenesis within the area of tissue.

Monocytes for use in the invention can be purified from natural sources, including blood, bone marrow and circulating progenitor cells. In one embodiment, they are prepared directly as differentiated monocytes from various sources, such as blood. In another embodiment, they are prepared from blood or bone marrow progenitors with appropriate growth factors and culture conditions. Monocytes can be expanded and differentiated from such progenitor cells, before or after purification, by exposure to factors, such as Macrophage-Colony Stimulating Factor (M-CSF) and GM-CSF. Suitable processes for purifying monocytes from such sources include, for example, immunopurification methods using antibodies which bind to surface antigens on monocytes, such as CD14, or which bind to surface antigen on other immune cells but not monocytes, such as CD2, CD3, CD19, CD56, CD66b, glycophorin A, so that monocytes can be negatively selected for.

In one embodiment, monocytes used in the present invention are activated, so as to produce particular endogenous proteins, prior to their being contacted with localized tissue to promote angiogenesis. This can be achieved by exposing the monocytes to purified activating proteins, such as GM-CSF, MCP-1, MCP-2, MCP-3, MCP-4, Interferon-γ, and Platelet Activating Factor (PAF). Alternatively, this can be achieved by transfecting the monocytes with one or more expression vectors encoding such activating proteins, so that the activating proteins are expressed by the monocytes. Suitable expression vectors include, for example, adenoviral vectors, retroviral vectors, lentiviral vectors, adeno-associated virus (AAV), naked DNA, transposons and a variety of other RNA and DNA vectors. Suitable transfection methods include, for example, liposomal transfection, transfection mediated by DEAE dextran, electroporation, and calcium phosphate precipitation.

In another embodiment, monocytes used in the present invention are transformed to express one or more therapeutic angiogenesis-promoting proteins, prior to being contacted with localized tissue, such that the therapeutic proteins are expressed and secreted by the monocytes, along with natural endogenous therapeutic angiogenesis promoting proteins. In the case where the monocytes are expanded and differentiated, this transfection step can take place before or after such expansion and differentiation. Suitable therapeutic proteins for promoting angiogenesis include, for example, M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, and Endothelin-1.

In another aspect, the invention provides a method of promoting angiogenesis in a subject by contacting a localized area of tissue within the subject with a monocyte chemoattractant, such that endogenous monocytes are recruited to, and accumulate at, the tissue area. Suitable chemoattractants include, for example, GM-CSF, Macrophage Inflammatory Protein 1-α (MIP-1α), Macrophage Inflammatory Protein 1-β (MIP-1β), Monocyte Chemotactic Protein (MCP)-1, MCP-2, MCP-3, MCP-4 and the Regulated upon Activation, Normal T cell Expressed and presumably Secreted (RANTES) protein. In one embodiment, this is achieved by directly administering the chemoattractant to the localized tissue area in an amount suitable to cause accumulation of an appropriate number of monocytes for promoting angiogenesis. In another embodiment, this is achieved by transforming cells at the localized tissue area to express the chemoattractant.

Methods and monocyte compositions of the present invention can be used to promote angiogenesis in a safe and controlled manner in a variety of selected localized tissue areas. Accordingly, such methods and compositions can be used to treat a variety of tissue ischemias, including myocardial ischemia, ischaemic bowel disease, and peripheral ischemia.

### Detailed Description of the Invention

The depletion of oxygen supply to due to obstructed or inadequate blood supply is the common pathological state associated with tissue ischemia, including myocardial ischemia, ischaemic bowel disease, and peripheral ischemia. The alleviation of the ischemic condition, and its attendant pathologies such as hypoxia, is critically dependant upon the process of angiogenesis, whereby new capillaries are generated from existing vasculature. The cellular process of angiogenesis can be artificially induced or even enhanced by application of therapeutic proteins. This is optimally achieved by sustained application of such proteins for not more than 2-3 months. However, current methods for local drug delivery fail to achieve this goal.

The present invention addresses this problem using monocytes to provide sustained, controlled local delivery of therapeutic proteins. According to the methods of the invention, monocytes, which naturally produce angiogenesis-inducing proteins and which can be transformed to express additional angiogenesis-inducing proteins, and which generally have a lifespan of approximately 2-3 months, are contacted with, or recruited to, selected tissue areas where they secrete such proteins to promote angiogenesis.

Monocytes represent an important component of the immune system by playing a role in antigen presentation and by providing an important source of growth factors and immunomodulatory signaling peptides, including several cytokines. Monocytes can be naturally or deliberately induced into an “activated” state in which they produce such proteins by exposure to activating factors, a number of which are well know in the art (e g., GM-CSF, MCP-1, MCP-2, MCP-3, MCP-4, Interferon-γ, and/or Platelet Activating Factor (PAF)). Alternatively, they can be activated by transformation with expression vectors which encode such activating factors.

“Monocytes” include all white blood cells originating from pluripotent stem cells in bone marrow (see, for example, Alberts et al, Molecular Biology of the Cell, Chapter 17, pp 973-982, Garland Publishing, 1994). These pluripotent cells must first differentiate into committed progenitor cells, then into monocytes. Monocytes can also further differentiate into other effector cells, such as macrophages. Accordingly, as used herein, the term “monocytes” includes not only differentiated monocytes, but also pluripotent stem cell and committed progenitor cells which differentiate into monocytes, as well as other effector cells which terminally differentiate from monocytes. On a functional level, monocytes are involved in inflammation and are able to specifically detect certain chemmoattractants (e.g., chemokines) which cause them to migrate directionally in response to gradients of such chemoattractants (e.g., chemotaxis).

Suitable and preferred sources of monocytes for use in the invention include blood, immortal cell lines, and pluripotent stem cells which can be derived from bone marrow, expanded and cultured. Monocytes can also be derived from circulating committed progenitor cells that have left the bone marrow but have not yet differentiated into monocytes. Pluripotent cells and committed progenitor cells can be cultured and treated (i.e., contacted with appropriate growth factors) such that they expand and differentiate into monocytes. Thus, monocytes for use in the invention can be purified in differentiated form or in undifferentiated form followed by ex vivo differentiation.

In either case, monocytes can be purified using a number of suitable techniques which are well known in the art. Such techniques include, for example, buffy-coat (BC) depletion, centrifigation, fluorescence-activated cell sorting (FACS), immunoprecipitation and generation of peripheral blood fractions. In a preferred embodiment, monocytes of the invention are purified using antibodies based on the presence or absence of specific cell-surface proteins. Antibodies against such antigens can be used to enrich a population of monocytes and to deplete a sample of non-monocyte cells. Preferred cell-surface proteins for such purification strategies include markers which are not present on monocytes, such as CD2, CD3, CD19, CD56, CD66b, and glycophorin A (e.g., in a method of negative selection). Other preferred cell-surface markers for such purification strategies include markers which are present on monocytes, such as CD14 (e.g., in a method of positive selection).

Monocytes for use in the present invention also can be activated, so as to express certain proteins which are normally not expressed when the monocytes are in an unactivated state, before or after they are purified. This can be achieved by exposure to one or more activating proteins, e.g., GM-CSF, MCP-1, MCP-2, MCP-3, MCP-4, Interferon-γ, and/or Platelet Activating Factor (PAF), preferably in purified form. Alternatively, monocytes can be activated by transfection with one or more expression vectors which encode such activating proteins. Those skilled in the art are readily able to prepare such expression vectors, such that functional activating proteins are expressed and secreted by the monocytes. In such embodiments, the monocytes themselves can be transformed to express the activating protein, or cells present at the tissue area to be treated (e.g., fibroblasts) can be transformed to express the activating protein so that monocytes delivered or recruited to the tissue area are exposed to the activating protein.

Suitable expression vectors for transforming monocytes or other tissue cells in accordance with the embodiments described herein are well known in the art and include, for example, adenoviral vectors, retroviral vectors, lentiviral vectors, adeno-associated virus (AAV) vectors, naked DNA vectors, transposons and a variety of other suitable RNA and DNA vectors (see, for example, Chapter 9 of Ausubel et al, Current Protocols in Molecular Biology, John Wiley & Sons, N.Y. (1989)). Methods for introducing these vectors into monocytes and other tissue cells are also well known in the art. For example, transfection techniques which utilize liposomes, cationic lipids, DEAE dextran, electroporation, calcium phosphate/nucleic acid precipitates (see, for example, Chapter 9 of Ausubel et al Current Protocols in Molecular Biology, John Wiley & Sons, N.Y. (1989)), and gene guns (e.g., Bio-Rad) can be used.

Monocytes for use in the invention, particularly activated monocytes, produce and secrete therapeutic proteins once delivered or recruited to a localized tissue area. As used herein, “therapeutic proteins” include proteins, factors, peptides and small molecule compounds which are able to induce or enhance angiogenesis. These include both natural endogenous therapeutic proteins expressed by monocytes, as well as therapeutic proteins produced recombinantly by monocytes. Accordingly, in one embodiment of the invention, monocytes are transformed to express additional selected therapeutic proteins which they do not naturally express prior to their delivery or recruitment to localized tissue areas. Suitable therapeutic proteins include, for example, M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, Endothelin-1 and combinations thereof.

The term “angiogenesis” refers to the generation of new blood supply, e.g., blood capillaries, vessels, and veins, from existing blood vessel tissue (e.g., vasculature). The process of angiogenesis can involve a number of tissue cell types including, for example, endothelial cells which form a single cell layer lining of all blood vessels and are involved with regulating exchanges between the bloodstream and the surrounding tissues. New blood vessels (angiogenesis) can develop from the walls of existing small vessels by the outgrowth of endothelial cells.

Following purification and, optionally transfection and/or activation, monocytes used in the invention are locally delivered to preselected tissue areas, for example, ischemic areas. This can be achieved using a number of suitable methods known in the art including, e.g., mechanical methods, such as surgical implantation. In a preferred embodiment, the cells are injected into a selected tissue area in an amount sufficient to promote or enhance angiogenesis.

In another embodiment, endogenous monocytes are recruited to the selected area of treatment by local delivery of chemoattractants to the area. As used herein, “chemoattractants” include proteins and bioactive molecules which cause monocyte chemotaxis, migration and accumulation in the area of the delivered chemoattractant. Such chemoattractants can be delivered to the area, for example, by injection. Alternatively, tissue cells within the area of treatment can be transformed to express chemoattractants using known transfection (e.g., gene delivery) techniques so that monocytes are recruited to the area.

Following recruitment or delivery to a localized tissue area, mono cytes of the invention secrete therapeutic proteins which promote angiogensis. Accordingly, methods of the invention can be used to treat a variety of tissue ischemias, including myocardial and peripheral tissue ischemia.

This invention is further illustrated by the following examples which should not be construed as limiting. The contents of all references, patents and published patent applications cited throughout this application are incorporated herein by reference.

### EXAMPLES

### Example 1

Alleviation of Tissue Ischemia by Direct Application of Purified Monocytes

White blood cells are separated from whole blood using standard techniques for generating peripheral blood available to one skilled in the art. Whole blood can be supplied by the subject, or can be obtained from a blood bank. Further purification of monocytes from other white blood cells can be accomplished by exploiting antibodies against cell markers that are specific for monocytes (e.g., CD14) or specific for nonmonocytes (e.g., CD2 CD3 CD19, CD56, CD66b, glycophorin A). The antibodies can be used to purify monocytes, for example, by conjugating the antibodies directly (via covalent modification, e.g cross-linking reactions) or indirectly (e.g. via immobilized protein A or protein G) to an inert matrix (e.g. agarose, cellulose). Immobilized on such matrix material, antibodies can be used to separate monocytes from non-monocytes through immunoprecipitation, sedimentation, or similar methods (see, for example, Chapter 14 of Coligan et al, Current Protocols in Immunology, John Wiley & Sons, N.Y. (1999)).

Monocytes that have been isolated in this way can then be introduced directly into sites of tissue ischemia by injection. Once introduced into the tissue, therapeutic proteins which induce angiogenesis are produced and secreted by the monocytes.

### Example 2

Alleviation of Tissue Ischemia by Direct Application of Cultured Monocytes

Pluripotent stem cells are harvested from the bone marrow or bone marrow samples using techniques known in the art (e.g., stem cell apheresis). These pluripotent cells can be cultured for a period of time and, during that time, treated with appropriate differentiation and growth factors such that the cells differentiate first into committed progenitor cells, then into monocytes. The profile and concentration of growth factors used, as well as the timing of their usage, is important to ensure that the pluripotent stem cells do not differentiate into lymphoid cells, or, alternatively, that committed progenitor cells do not differentiate into erythrocytes, granulocytes, or most importantly, neutrophils.

Alternatively, committed progenitor cells can be isolated from circulating blood by techniques described herein. Committed progenitor cells have originated from pluripotent stem cells which have been set on a differentiation path that is directed toward the committed progenitor lineage (as opposed to a lymphoid lineage). Like pluripotent stem cells, the progenitor cells can be cultured and differentiated into monocytes when the culture medium is supplemented with appropriate concentrations and profiles of growth factors. The successfully differentiated culture of monocytes can be distinguished by microscopic evaluation of live cells by one skilled in the art, or by differential staining.

Monocytes can then be delivered directly (e.g., injected) into tissue in which angiogenesis is desired (e.g. ischemic tissue).

### Example 3

Activation of Monocytes by Exposure to Activating Proteins

Some therapeutic proteins are not produced and/or secreted by monocytes when they are in an non-activated state. Thus, in some instances, it is beneficial to activate them by exposing them to activating proteins. To achieve this, monocytes can be cultured in appropriate media after purification from peripheral blood and exposed to activating proteins while in culture. Activating proteins that are known in the art can be purchased or obtained by one skilled in the art and added directly to the monocyte culture medium.

Alternatively, activation of these cultured monocytes can be accomplished by introduction of exogenous activation genes into the monocytes (e.g., transfection). Activation genes can be genes which encode complete or partial peptides which are known in the art to activate monocytes, e.g., GM-CSF, MCP-1, Interferon-γ, and Platelet Activating Factor (PAF). One or more activation genes can be incorporated onto appropriate expression vectors and introduced into the cultured cells using any standard transfection method. Monocytes are then be cultured for an appropriate time until they become activated from the expression of exogenous activation gene product.

Activated monocytes can then be introduced (injected) directly into sites of tissue ischemia.

### Example 4

Enhancing Monocyte-mediated Angiogenesis by Transfection with Genes for Therapeutic Proteins

Monocytes that have been purified from whole blood or, alternatively, that have been differentiated from harvested pluripotent bone marrow stem cells or from committed progenitor cells (isolated from whole blood) can be cultured in vitro prior to introduction into areas of tissue ischemia. In order to boost the intrinsic angiogenic effect of proteins which the monocytes naturally express, genes which encode additional therapeutic proteins (angiogenic factors) can be introduced into the cultured monocytes prior to injection of the cells into ischemic tissue. This requires the use of an appropriate expression vector, as well as an effective transfection methodology, both of which can be chosen by those of skill in the art. Suitable genes for transfection include, for example, M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, and Endothelin-1. Any one of these genes can be introduced into cultured monocytes to enhance their angiogenic effect prior to their delivery or recruitment to localized areas.

### Example 5

Using a Subject's Endogenous Monocyte Population to Induce Localized Angiogenesis

Monocytes have the ability to migrate along gradients of certain chemoattractant molecules, such as chemokines (e.g., chemotaxis). Monocyte-mediated angiogenesis can be induced by localization of endogenous monocytes in an area where angiogenesis is desired by exploiting this cellular property. This can be accomplished by introducing monocyte-specific chemoattractant peptides or molecules directly into the site of tissue ischemia and allowing endogenous monocytes move toward the area where angiogenesis is desired. Natural or recombinant angiogenic factors are then produced and secreted by monocytes that have moved into the desired area through chemotaxis.

Equivalents

Those skilled in the art will recognize, or be able to ascertain using no more than routine experimentation, many equivalents of the specific embodiments of the invention described herein. Such equivalents are intended to be encompassed by the following claims.

What is claimed is:

1. A method of promoting angiogenesis comprising contacting a localized area of tissue with purified monocytes in an amount effective to induce angiogenesis within the area of tissue.

2. The method of claim 1, wherein the monocytes are purified from a source selected from the group consisting of blood, bone marrow and circulating progenitor cells.

3. The method of claim 2, wherein the monocytes are expanded and differentiated by exposure to a factor selected from the group consisting of Macrophage-Colony Stimulating Factor (M-CSF) and GM-CSF.

4. The method of claim 3, wherein the expansion and differentiation is effected prior to purification of the monocytes.

5. The method of claim 2, wherein the monocytes are purified using one or more antibodies selected from the group consisting of CD2, CD3, CD19, CD56, CD66b, glycophorin A, and CD14.

6. The method of claim 1, wherein the purified monocytes are activated prior to being contacting with the area of tissue.

7. The method of claim 6, wherein the monocytes are activated by exposure to one or more purified activating proteins.

8. The method of claim 7, wherein activation is effected by contacting the monocytes with a compound selected from the group consisting of MCP-2, MCP-3, MCP-4, GM-CSF, MCP-1, Interferon-γ, and Platelet Activating Factor (PAF).

9. The method of claim 6, wherein activation is effected by transfecting the monocytes with one or more expression vectors encoding an activating protein.

10. The method of claim 9, wherein the one or more expression vectors are selected from the group consisting of adenoviral vectors, retroviral vectors, RNA vectors, DNA vectors, naked DNA, lentiviral vectors, adeno-associated virus (AAV), and transposons.

11. The method of claim 9, wherein transfection is effected by a method selected from the group consisting of liposomal transfection, transfection mediated by DEAE dextran, electroporation, and calcium phosphate precipitation.

12. The method of claim 9, wherein the activating protein is selected from the group consisting of MCP-2, MCP-3, MCP-4, GM-CSF, MCP-1, Interferon-γ, and Platelet Activating Factor (PAF).

13. The method of claim 1, wherein the monocytes are transformed to express one or more therapeutic proteins prior to being contacted with the area of tissue.

14. The method of claim 3, wherein the monocytes are transformed to express one or more therapeutic proteins prior to being expanded and differentiated.

15. The method of claim 3, wherein the monocytes are transformed to express one or more therapeutic proteins after being expanded and differentiated.

16. The method of claim 13, wherein the monocytes are transformed with an expression vector selected from the group consisting of adenoviral vectors, retroviral vectors, RNA vectors, DNA vectors, naked DNA, lentiviral vectors, adeno-associated virus (AAV), and transposons.

17. The method of claim 16, wherein the monocytes are transformed using a method selected from the group consisting of liposomal transfection, transfection mediated by DEAE dextran, electroporation, and calcium phosphate precipitation.

18. The method of claim 13, wherein the therapeutic protein is selected from the group consisting of M-CSF, GM-CSF, VEGF-A, VEGF-B, VEGF-C, VEGF-D, basic FGF, PDGF-B, Angiopoietin 1, Angiopoietin 2, erythropoietin, BMP-2, BMP-4, BMP-7, TGF-beta, IGF-1, Osteopontin, Pleiotropin, Activin, and Endothelin-1.

19. The method of claim 1, wherein the area of tissue contacted with the monocytes is ischemic.

20. A method of promoting angiogenesis in a subject comprising contacting a localized area of tissue within the subject with a monocyte chemoattractant, such that endogenous monocytes accumulate at the tissue area.

21. The method of claim 22, wherein the chemoattractant is selected from the group consisting of GM-CSF, Macrophage Inflammatory Protein 1-α (MIP-1α), Macrophage Inflammatory Protein 1-β (MIP-1β), Monocyte Chemotactic Protein (MCP) MCP-1, MCP-2, MCP-3, MCP-4 and the Regulated upon Activation, Normal T cell Expressed and presumably Secreted (RANTES) protein.

22. The method of claim 20, wherein tissue cells at the localized area are transformed to express the chemoattractant.

23. The method of claim 22, wherein the cells are transformed with an expression vector selected from the group consisting of adenoviral vectors, retroviral vectors, RNA vectors, DNA vectors, naked DNA, lentiviral vectors, adeno-associated virus (AAV), and transposons.

24. The method of claim 23, wherein the cells are transformed using a method selected from the group consisting of liposomal transfection, transfection mediated by DEAE dextran, electroporation, and calcium phosphate precipitation.
